(19) United States
(12) Patent Application Publication
(10) Pub. No.: US 2025/0073258 A1
(43) Pub. Date: Mar. 6, 2025

(54) Compositions and Methods to Protect and Enhance Structural and Functional Integrity of the Intestinal Glycocalyx

(71) Applicants: Calroy Health Sciences, LLC, Scottsdale, AZ (US); HealthSyn Biotechnology (Beijing) Co., Ltd., Beijing (CN); Morningbell Wellness, LLC, Greensboro, NC (US); Zhongke Runxin (Suzhou) Biological Technology Co., Ltd., Suzhou (CN)

(72) Inventors: Chen CHEN, Scottsdale, AZ (US); Edward HOYT, Scottsdale, AZ (US); Jianjun LI, Beijing (CN); Kevin CHEN, Scottsdale, AZ (US); Lishi REN, Suzhou (CN); Ming SUN, Suzhou (CN); Siming JIAO, Beijing (CN); Yuchen ZHANG, Beijing (CN); Yucheng DU, Scottsdale, AZ (US); Yuguang DU, Suzhou (CN); Zhuo WANG, Suzhou (CN)

(21) Appl. No.: 18/937,981

(22) Filed: Nov. 5, 2024

Related U.S. Application Data
(63) Continuation of application No. 18/817,800, filed on Aug. 28, 2024, now abandoned.
(60) Provisional application No. 63/579,240, filed on Aug. 28, 2023.
(60) Provisional application No. 63/579,249, filed on Aug. 28, 2023.

(51) Int. Cl. A61K 31/722; A61K 31/702; A61K 31/737; A61K 36/185; A61P 1/00

(52) U.S. Cl. CPC A61K 31/722; A61K 31/702; A61K 31/737; A61K 36/185; A61P 1/00

(57) ABSTRACT

Compositions and methods are provided herein for treating intestinal glycocalyx dysfunction. The composition can comprise a chito-oligosaccharide (COS), and optionally a human milk oligosaccharide (hMO) in an amount sufficient to maintain an intestinal glycocalyx and a pharmaceutically acceptable carrier. The composition can be formulated as an oral dosage form or a parenteral dosage form.

The method can comprise identifying intestinal glycocalyx dysfunction in the subject. The method can further comprise administering to the subject a COS in an amount and at a frequency sufficient to prevent, stabilize and reverse damage in the intestinal glycocalyx.

### RELATED APPLICATION

This application is a continuation of U.S. patent application Ser. No. 18/817,800, filed Aug. 28, 2024, which claims priority to U.S. Provisional Patent Application No. 63/579,240, filed Aug. 28, 2023 and U.S. Provisional Patent Application No. 63/579,249, filed Aug. 28, 2023, which are each incorporated herein by reference.

### BACKGROUND

The human intestine tract is lined with a rapidly renewing monolayer of epithelial cells. The intestinal cells are exposed to a harsh environment including mechanical stresses arising from luminal content propulsion along the intestinal tract. To protect the intestinal tract from these mechanical, biomedical, and pathogen stresses, epithelial cells generate a protective glycocalyx layer that covers the surface of epithelial cells and acts as a semi-permeable barrier between the lumen and the epithelium. The intestinal glycocalyx comprises glycosaminoglycans, proteoglycans, glycoproteins and glycolipids that can be receptors for bacterial adhesion. That is, the glycocalyx can be attached to normal flora to limit pathogenic threats and exclude harmful bacteria and viruses in addition to behaving as a size selective diffusion barrier. The intestinal glycocalyx also participates in other functions. Because of its role in limiting pathogenic threats, dysfunction in the intestinal glycocalyx can have deleterious effects.

### SUMMARY

In one embodiment, a therapeutic composition for treating intestinal glycocalyx dysfunction can comprise a chito-oligosaccharide (COS) in an amount sufficient to maintain an intestinal glycocalyx, and a pharmaceutically acceptable carrier. In one example, the composition can further comprise at least one of a human milk oligosaccharide (hMO) and a rhamnan sulfate.

In another embodiment, an oral dosage form can comprise a chito-oligosaccharide (COS) in an amount sufficient to maintain an intestinal glycocalyx, and a pharmaceutically acceptable carrier.

In another embodiments, a parenteral dosage form can comprise a chito-oligosaccharide (COS) in an amount sufficient to maintain an intestinal glycocalyx, and a pharmaceutically acceptable carrier.

In yet another embodiment, a method of treating intestinal glycocalyx dysfunction in a subject can comprise identifying intestinal glycocalyx dysfunction in the subject, and administering to the subject a chito-oligosaccharide (COS) in an amount and at a frequency sufficient to stabilize and reverse damage in an intestinal glycocalyx.

There has thus been outlined, rather broadly, the more important features of the disclosure so that the detailed description thereof that follows may be better understood, and so that the present contribution to the art may be better appreciated. Other features of the present disclosure will become clearer from the following detailed description of the disclosure, taken with the accompanying drawings and claims, or may be learned by the practice of the disclosure.

### BRIEF DESCRIPTION OF THE DRAWINGS

FIG. 1 are images of Caco-2 intestinal epithelial cell glycocalyx with different treatments after immunofluorescence staining in accordance with one example.

FIG. 2 shows a bar chart of the average florescence intensity of Caco-2 intestinal epithelial cell glycocalyx after WGA-FITC staining in accordance with another example.

FIG. 3 is a graph of mouse body weight change after DSS treatment in accordance with another example.

FIG. 4 is a graph of mouse body weight change at the end of the study in accordance with another example.

FIG. 5 is a graph of mouse DAI after DSS treatment in accordance with another example.

FIG. 6 is a graph of mouse DAI at the end of the study in accordance with another example.

FIG. 7 are images of histological structure of mouse colon after HE staining in accordance with yet another example.

FIG. 8 are images of mouse intestinal glycocalyx with immunofluorescence staining in accordance with another example.

FIG. 9 is a bar graph of average florescence intensity of mouse intestinal glycocalyx after WGA-FITC staining in accordance with another example.

### DETAILED DESCRIPTION

While these exemplary embodiments are described in sufficient detail to enable those skilled in the art to practice the disclosure, it should be understood that other embodiments may be realized and that various changes to the disclosure may be made without departing from the spirit and scope of the present disclosure. Thus, the following more detailed description of the embodiments of the present disclosure is not intended to limit the scope of the disclosure, as claimed, but is presented for purposes of illustration only and not limitation to describe the features and characteristics of the present disclosure, to set forth the best mode of operation of the disclosure, and to sufficiently enable one skilled in the art to practice the disclosure. Accordingly, the scope of the present disclosure is to be defined solely by the appended claims.

### Definitions

In describing and claiming the present disclosure, the following terminology will be used.

The singular forms “a,” “an,” and “the” include plural referents unless the context clearly dictates otherwise. Thus, for example, reference to “a cofactor” includes reference to one or more of such materials and reference to “subjecting” refers to one or more such steps.

As used herein, the term “human milk oligosaccharide” or “hMO” refers to any one of the more than 200 such structures known to be present in human milk.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

The term “dosage unit” or “dose” are understood to mean an amount of an active agent that is suitable for administration to a subject in order achieve or otherwise contribute to a therapeutic effect. In some examples, a dosage unit can refer to a single dose that is capable of being administered to a subject or patient, and that may be readily handled and packed, remaining as a physically and chemically stable unit dose.

As used herein, a “dosing regimen” or “regimen” such as “treatment dosing regimen,” or a “prophylactic dosing regimen” refers to how, when, how much, and for how long a dose of an active agent or composition can or should be administered to a subject in order to achieve an intended treatment or effect.

As used herein, “daily dose” refers to the amount of active agent (e.g., chito-oligosaccharide (COS)) administered to a subject over a 24-hour period of time. The daily dose can be administered in two or more administrations during the 24-hour period. With this in mind, an “initial dose” or initial daily dose” refers to a dose administered during the initial regimen or period of a dosing regimen.

As used herein, “solid” and “semi-solid” refers to the physical state of a composition that supports its own weight at standard temperature and pressure and has adequate viscosity or structure to not freely flow. Semi-solid materials may conform to the shape of a container under applied pressure.

As used herein, the term “particulate-matter-free”, “particulate free,” or “particle free” refer to a state in which the composition of the present disclosure meets the USP standards for particulate matter in parenteral solutions. See e.g., United States Pharmacopeia (USP), <788>. One of skill in the art understands and knows how to assess whether a given composition meets USP particulate matter standards.

As used herein, the terms “treat,” “treatment,” or “treating” refers to administration of a therapeutic agent to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition.

As used herein, the terms “therapeutic agent,” “active agent,” and the like can be used interchangeably and refer to an agent that can have a beneficial or positive effect on a subject when administered to the subject in an appropriate or effective amount. In one aspect, the therapeutic or active agent can be a chito-oligosaccharide (COS). The terms “additional active agent,” “supplemental active agent,” “secondary active agent,” and the like can be used interchangeably and refer to a compound, molecule, or material other than a COS that has physiologic activity when administered to a subject in an effective amount.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients. Furthermore, the term “dosage form” can include one or more formulation(s) or composition(s) provided in a format (e.g., a specific form, shape, vehicle, etc.) for administration to a subject. For example, an “oral dosage form” can be suitable for administration to a subject's mouth. In another example, a “parenteral dosage form” can be suitable for parenteral administration by subcutaneous administration, intraperitoneal administration, intradermal administration, intramuscular administration, intravenous, the like, or a combination thereof.

As used herein, “pharmaceutically acceptable carrier” or “carrier” are used interchangeably and refer to a pharmaceutical acceptable agent that can be capable of fully or partially dissolving or solubilizing an active agent (e.g., a COS) in the pharmaceutical composition. Further, in some aspects, the carrier can be an agent that can be varied for the alteration of release rate and/or extent of the active agent from the composition and/or the dosage form.

The phrase “effective amount,” “therapeutically effective amount,” or “therapeutically effective rate(s)” of an active ingredient refers to a substantially non-toxic, but sufficient amount or delivery rates of the active ingredient, to achieve therapeutic results in treating a disease or condition for which the drug is being delivered. It is understood that various biological factors may affect the ability of a substance to perform its intended task. Therefore, an “effective amount,” “therapeutically effective amount,” or “therapeutically effective rate(s)” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of a therapeutically effective amount or delivery rate is well within the ordinary skill in the art of pharmaceutical sciences and medicine.

As used herein, a “subject” refers to an animal. In one aspect the animal may be a mammal. In another aspect, the mammal may be a human.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term, like “comprising” or “including,” in the written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, comparative terms such as “increased,” “decreased,” “better,” “worse,” “higher,” “lower,” “enhanced,” “maximized,” “minimized,” and the like refer to a property of a device, component, composition, or activity that is measurably different from other devices, components, compositions or activities that are in a surrounding or adjacent area, that are similarly situated, that are in a single device or composition or in multiple comparable devices or compositions, that are in a group or class, that are in multiple groups or classes, or as compared to the known state of the art.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of” For example, “at least one of A, B and C” explicitly includes only A, only B, only C, and combinations of each.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the disclosure should be determined solely by the appended claims and their legal equivalents, rather than by the descriptions and examples given herein.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

In some examples, compositions and methods can include the compositions and methods disclosed in: (i) U.S. patent application Ser. No. 16/219,854, which issued as U.S. Pat. No. 11,135,238 on Oct. 5, 2021, and (ii) U.S. Pat. App. No. 17,735,106, which published as US-2022-033135 on Oct. 20, 2022, which are incorporated herein by reference.

### Compositions and Methods to Protect and Enhance Functional Integrity of the Intestinal Glycocalyx

An initial overview of disclosure embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

The epithelial glycocalyx layer comprises glycosaminoglycans, proteoglycans, glycoproteins and glycolipids that can exclude harmful pathogens, function in cellular signaling, and selectivity bar endogenous and exogenous substances. Some diseases associated with intestinal glycocalyx dysfunction include inflammatory bowel disease, leaky gut syndrome, and certain types of cancer.

The intestinal glycocalyx includes various transmembrane mucins, such as MUC1, MUC3, MUC4, MUC12, MUC13, and MUC17. The extracellular domains of these mucins have mucin tandem repeats that have O-glycosylation on the serine and threonine residues—a sugar modification that contributes to steric hindrance, negative charge repulsion, and increased hydrophilic regions and thereby affects mucin filament interaction and the mechanical properties of the glycocalyx layer. Changes in the degree of glycosylation of the epithelial glycocalyx layer have also been associated with various diseases.

Therefore, the preservation, reversal of damage, and restoration of the intestinal glycocalyx can treat diseases associated with intestinal glycocalyx dysfunction. However, identifying dysfunction in the intestinal glycocalyx can be difficult in vivo. As such, identifying intestinal glycocalyx dysfunction can be used for more precise treatment of intestinal glycocalyx dysfunction. Furthermore, compositions that can effectively repair the intestinal glycocalyx and treat associated diseases have not been identified.

The present disclosure describes several compositions, dosage forms, and methods that can be used to maintain or reverse damage to the intestinal glycocalyx to treat intestinal glycocalyx dysfunction and related conditions. In some examples, the compositions, dosage forms, and methods described herein can be used to restore the intestinal glycocalyx.

In one embodiment, a therapeutic composition for treating intestinal glycocalyx dysfunction can comprise a COS in an amount sufficient to maintain an intestinal glycocalyx, and a pharmaceutically acceptable carrier. In one example, the composition can further comprise at least one of a rhamnan sulfate (RS), a human milk oligosaccharide (hMO), and a combination thereof. The composition can be formulated as an oral dosage form or a parenteral dosage form.

In yet another embodiment, a method of treating intestinal glycocalyx dysfunction in a subject can comprise identifying intestinal glycocalyx dysfunction in the subject. In one example, the method can comprise administering to the subject at least one of a COS, a rhamnan sulfate, and an hMO in an amount and at a frequency sufficient to stabilize and reverse damage in an intestinal glycocalyx.

### Compositions

In the present disclosure, it is noted that when discussing the compositions, the dosage forms, and the methods, each of these discussions can be considered applicable to each of these examples, whether or not they are explicitly discussed in the context of that example. Thus, for example, in discussing details about the compositions per se, such discussion also refers to the dosage forms and the methods described herein, and vice versa.

In more detail, a therapeutic composition for treating intestinal glycocalyx dysfunction can comprise a COS in an amount sufficient to maintain an intestinal glycocalyx. Chitosan is a biopolymer produced by the deacetylation of chitin, a major component of the shells of crustaceans. In some cases, the degree of deacetylation for chitosan can be 10% to 95%, and in some cases 50% to 95%. Chito-oligosaccharides are formed by depolymerizing chitin or chitosan using acid hydrolysis, hydrolysis by physical methods, and enzymatic degradation.

The degree of polymerization and molecular weight of the COS can be any suitable amount that can achieve the therapeutic effect. In one example, the COS can be a chitosan having a polymerization of less than or equal to 50, an average molecular weight of less than 10 kDa, or a combination thereof. In one example, the COS can have an average molecular weight of at least one of: from about 300 Da to about 3 kDa, from 500 Da to about 3 kDa, from about 3 kDa to about 5 kDa, from about 5 kDa to about 10 kDa, the like, and a combination thereof.

The COS can include any suitable COS including amino-derived COS, carboxlated COS, gallyl COS, sulfated COS, phenolic acid conjugated COS, the like, or a combination thereof. Amino-derived COS can include, but are not limited to, aminoethyl COS (AE-COS), dimethyl aminoethyl COS (DMAE-COS), diethyl aminoethyl COS (DEAE-COS), the like, or a combination thereof. Carboxylated COS can include compounds in which a carboxyl group (COCH2CH2COO—) has been introduced to the amino position of the pyranose unit. Gallyl COS can include compounds in which gallic acid has been conjugated with COS. Sulfated COS can include compounds in which sulfate has been substituted using any suitable sulfating reagent such as, but not limited to, chlorosulfonic acid, anhydrous formamide, trimethylamine sulfur trioxide, the like, or a combination thereof. Phenolic acid conjugated COS can include, but are not limited to, COS conjugated with at least one of: protocatechuic, 4-hydroxybenzoic, vanillic, syringic, p-coumaric, caffeic, ferulic, and sinapinic acid, the like, or a combination thereof.

The COS can be present in the composition in any suitable amount that can achieve a therapeutic effect. In one example, the COS can be present in the composition in an amount of from greater than 0 wt % to about 90 wt %, or about 0.0001 wt % to about 90 wt %. In another example, the COS can be present in the composition in an amount of from about 0.0001 wt % to about 50 wt %. In another example, the COS can be present in the composition in an amount of from about 0.0001 wt % to about 30 wt %. In yet another example, the COS can be present in the composition in an amount of from about 0.0001 wt % to about 20 wt %.

Several algal species can possess a variety of therapeutic properties. For example, some algal species include one or more bioactive polysaccharides, such as sulfated polysaccharides. These bioactive polysaccharides can have a variety of therapeutic properties, such as antioxidant, antitumor, immunomodulatory, anti-inflammatory, anticoagulation, anti-obesity, antiviral, antiprotozoan, antibacterial, antilipemic, or other bioactive properties. Non-limiting examples of marine algae polysaccharides, including sulfated polysaccharides, can include fucans, fucoidans, carrageenans, furcellaran, ulvans (e.g., rhamnan sulfate), galactans, or the like. In some examples, the composition can include at least one of a sulfated fucan, a fucoidan, a carrageenan, an ulvan, and a sulfated galactan. In some examples, the sulfated polysaccharide can include rhamnan sulfate, fucoidan sulfate, arabinan sulfate, arabinogalactan sulfate, galactan sulfate, mannan sulfate, the like, functional analogues thereof, or a combination thereof.

In some examples, the composition can include rhamnan sulfate. In some examples, the composition can include a fucoidan sulfate. In some examples, the composition can include an arabinan sulfate. In some examples, the composition can include an arabinogalactan sulfate. In some examples, the composition can include a galactan sulfate. In some examples, the composition can include a mannan sulfate. In some examples, functional analogues can include natural or synthetic oligosaccharides and polysaccharides. Non-limiting examples of functional analogues can include rhamno-oligosaccharides, fuco-oligosaccharides, galacto-oligosaccharide, fructo-oligosacchrides, sulfated rhamno-oligosaccharides, sulfated fuco-oligosaccharides, beta-glucans zylo-oligosaccharides, mannan oligosaccharides galacto-mannan-oligosaccharides, rhamnan sulfate oligosaccharides, heparan sulfate oligosaccharides, chondroitin sulfate oligosaccharides, keratan sulfate oligosaccharides, a non-digestible carbohydrate (NDC) such as an inulin (e.g., having a suitable degree of polymerization, e.g., 3 to 10, 10 to 60, or 30 to 60), a pectin having a suitable degree of methylation (e.g., 7, 55, 69, or the like), and the like.

It is noted that different varieties of fucans, fucoidans, carrageenans, furcellaran, ulvans, galactans, and the like can be extracted or derived from different species of marine algae. Thus, for example, a fucoidan derived from different species of brown algae may be somewhat different. Accordingly, sulfated polysaccharides derived or extracted from one species may have more desirable properties than a similar sulfated polysaccharide derived or extracted from another species within the same genus of algae.

Consequently, in some examples, the sulfated polysaccharide can be extracted or derived from red algae, brown algae, green algae, microalgae, or a combination thereof. In some examples, the sulfated polysaccharide can be extracted or derived from red algae. In some examples, the sulfated polysaccharide can be extracted or derived from brown algae. In some examples, the sulfated polysaccharide can be extracted or derived from green algae. In still other examples, the sulfated polysaccharide can be extracted or derived from microalgae. In some specific examples, the sulfated polysaccharide can include a polysaccharide extracted or derived from algae selected from the group consisting of Monostroma nitidum, Monostroma latissimum, Monostroma angicava, Ulva lactuca, Enteromorpha intestinalis, Caulerpa spp., Codium spp., Fucus spp., Sargassum vulgare, Sargassum fusiforme, Ecklonia cava, Ecklonia kurome, Laminaria spp., Chondrus crispus, Phyllophora brodiei, Grateloupia indica, Amphora coffeaeformis, Codium spp., and combinations thereof. In some additional examples, the sulfated polysaccharide can be or include a polysaccharide extracted or derived from Monostroma nitidum.

The composition and molecular weight of marine polysaccharides and oligosaccharides can vary with sources. However, they can generally be extracted with a hot water solution following certain pretreatment such as cleaning, drying, milling, demineralization, alkaline treatment, enzymatic treatment, or the like. The extracted polysaccharides may be further purified by separation columns and membranes such as size-exclusion chromatography and ion exchange chromatography. Any suitable method of extracting or deriving a bioactive polysaccharide from marine algae can be used to obtain a sulfated polysaccharide. Alternatively, some suitable equivalents, such as sulfated oligosaccharides, can be synthesized rather than extracted from natural sources.

In other examples, the composition can further comprise a sulfated polysaccharide, such as rhamnan sulfate. The sulfated polysaccharide (e.g., rhamnan sulfate), or a pharmaceutically acceptable salt or metal complex thereof, can be present in an amount sufficient to treat intestinal glycocalyx dysfunction.

The sulfated polysaccharide, such as rhamnan sulfate, can be present in the composition in any suitable amount that achieves a therapeutic effect. In one example, the sulfated polysaccharide (e.g., rhamnan sulfate) can be present in the composition in an amount of from greater than 0 wt % to about 90 wt %, or about 0.0001 wt % to about 90 wt %. In another example, the sulfated polysaccharide (e.g., rhamnan sulfate) can be present in the composition in an amount of from about 0.0001 wt % to about 50 wt %. In another example, the sulfated polysaccharide (e.g., rhamnan sulfate) can be present in the composition in an amount of from about 0.0001 wt % to about 30 wt %. In yet another example, the sulfated polysaccharide (e.g., rhamnan sulfate) can be present in the composition in an amount of from about 0.0001 wt % to about 20 wt %. Unless otherwise indicated, all percentages described herein refer to the amount of the component as a percentage of the total amount of the composition.

The molecular weight of the sulfated polysaccharide can impact absorption in the intestines and also the therapeutic effect on intestinal glycocalyx dysfunction. When the sulfated polysaccharide is rhamnan sulfate, the rhamnan sulfate can have any suitable average molecular weight to achieve adequate absorption and therapeutic effect. In one example, the rhamnan sulfate can have an average molecular weight of from about 50 kDa to about 2 MDa to achieve a therapeutic effect, or less than 500 kDa. In another example, the rhamnan sulfate can have an average molecular weight of from about 100 kDa to about 1 MDa. In another example, the rhamnan sulfate can have an average molecular weight of greater than 50 kDa, or from about 50 kDa to about 500 kDa, and in some cases to about 100 kDa.

In additional examples, the composition can further comprise a human milk oligosaccharide (hMO). In one example, the hMO can be a neutral hMO, a fucosylated hMO, a neutral N-containing hMO, a non-fucosylated hMO, an acid hMO, a sialylated hMO, the like, or a combination thereof. hMOs can include, but are not limited to, one or more of 2′-fucosyllactose (2′-FL), 3-fucosyllactose (3-FL), lactodifucotetraose (LD), lacto-N-tetraose (LNT), lacto-N-neotetraose (LNnT), lacto-N-fucopentaose I (LNF-I), lacto-N-fucopentaose II (LNF-II), lacto-N-difucohexaose I (LND-I), lacto-N-difucohexaose II (LND-II), 3′-sialyllactose (3′-SL), 6′-sialyllactose (6′-SL), 3′-Sialyl-N-acetyllactosamine (3′SLN), 6′-sialyl-N-acetyllactosamine (6′SLN), LST-a, LST-b, LST-c, 6′-galactosyllactose (6′-GaIL), lacto-N-fucopentaose V (LNF-V), lacto-N-fucopentaose III (LNF-III), lacto-N-hexaose (LNH), lacto-N-neohexaose (LNnH), 3′-galactosyllactose (3′-GaIL), 3-fucosyl-3′-SL (3F-3′-SL), sialyl-LNF-II (S-LNF-II), siayly-LNF-I (S-LNF-I), disialyl-LNT (DS-LNT), disialyl-LNF-II (DS-LNF-II), disialyl, LNF-V (DS-LNF-V), lacto-N-neo-difucohexaose II (LnND-II), 3-fucosyl-LST c (3F-LST c), lacto-N-hexaose (LNH) containing hMOs, LNnH containing hMOs, para-Lacto-N-hexaose, para-Lacto-N-neohexaose, lacto-N-octaose, lacto-N-neooctaose, iso-Lacto-N-octaose, para-lacto-N-octaose, lacto-N-decaose, para-LNH containing hMOs, para-LNnH containing hMOs, lacto-N-octaose containing hMOs, lacto-N-neooctaose containing hMOs, iso-Lacto-N-octaose containing hMOs, the like, or a combination thereof. In some examples, the composition can comprise a non-2-fucosyllactose hMO.

The hMO can be present in the composition in any suitable amount to provide a therapeutic effect. In one example, the hMO can be present in the composition in an amount of from greater than 0 wt % to about 90 wt %, or about 0.0001 wt % to about 90 wt %. In another example, the hMO can be present in the composition in an amount of from about 0.0001 wt % to about 50 wt %. In another example, the hMO can be present in the composition in an amount of from about 0.0001 wt % to about 30 wt %. In another example, the hMO can be present in the composition in an amount of from about 0.0001 wt % to about 20 wt %.

In further examples, a composition for treating intestinal glycocalyx dysfunction can include a combination of at least one of a sulfated polysaccharide (e.g., rhamnan sulfate), a COS, and an hMO in an amount sufficient to maintain an intestinal glycocalyx. In one example, the composition can include a sulfated polysaccharide (e.g., rhamnan sulfate) and a COS. In another example, the composition can include a sulfated polysaccharide (e.g., rhamnan sulfate) and an hMO. In another example, the composition can include a sulfated polysaccharide (e.g., rhamnan sulfate), a COS, and an hMO. In another example, the composition can include a COS and an hMO.

The combination of the COS with the hMO can be present in the composition in a suitable weight ratio (w/w) for achieving the therapeutic effect. In one example, the weight ratio of COS to hMO can be from about 10:1 to about 1:10.

The combination of the sulfated polysaccharide with at least one of the COS and the hMO can be present in the composition in a suitable weight ratio (w/w) for achieving the therapeutic effect. In one example, the weight ratio of sulfated polysaccharide to COS can be from about 10:1 to about 1:20. In another example, the weight ratio of sulfated polysaccharide to hMO can be from about 10:1 to about 1:20. In yet other examples, the weight ratio of sulfated polysaccharide to COS to hMO can be at least one of: from about 1:1:1 to about 10:1:1; from about 1:1:1 to about 1:20:1; from about 1:1:1 to about 1:1:20; the like, or a combination thereof.

In some examples, the combination of the COS with at least one of the hMO and sulfated polysaccharide can be present in the composition in an amount sufficient to maintain or increase at least one of an intestinal glycocalyx thickness, an intestinal glycocalyx length, an intestinal glycocalyx volume, an intestinal glycocalyx integrity, and a combination thereof.

The composition can further comprise an intestinal glycocalyx precursor. The glycocalyx regenerator can be any suitable components of the intestinal glycocalyx. In some examples, the glycocalyx precursor can include, but is not limited to: a glycan selected from the group consisting of sialic acid, glucosamine, hyaluronic acid, chondroitin sulfate, heparan sulfate, dermatan sulfate or a combination thereof.

The glycocalyx precursor can be present in the composition in any suitable amount to provide a therapeutic effect. In one example, the glycocalyx precursor can be present in the composition in an amount of from about 0.0001 wt % to about 90 wt %. In another example, the glycocalyx precursor can be present in the composition in an amount of from about 0.0001 wt % to about 50 wt %. In another example, the glycocalyx precursor can be present in the composition in an amount of from about 0.0001 wt % to about 30 wt %. In yet another example, the glycocalyx presursor can be present in the composition in an amount of from about 0.0001 wt % to about 20 wt %.

In some examples, an antioxidant can have a synergistic effect when used in the composition. The composition can include any suitable antioxidant including butylated hydroxyanisole (BHA), butylated hydroxytoluene (BHT), ascorbic acid, ascorbyl palmitate, alpha-lipoic acid, N-acetyl cysteine, glutathione, carotenoids, coenzyme Q10, trans-resveratrol, tocopherols, tocotrienols, potassium metabisulfite, sodium thiosulfate, alliin, propyl gallate, epigallocatechin gallate, the like, or a combination thereof. In another example, the antioxidant can include superoxide dismutase (SOD), catalase, glutathione peroxidase, the like, or combinations thereof.

In other examples, the antioxidant can include a plant-based powder blend rich in antioxidants and prebiotics such as polyphenols. Plant-based powder may be produced by different technologies including a fermentation process. Antioxidant polyphenols can be effective at reducing oxidative stress and reactive oxygen species (ROS). Non-limiting examples of plant-based antioxidant-rich powders can include red grape skin extract, red grape seed extract, white grape skin extract, white grape seed extract, green tea extract, carrot juice or extract, tomato juice or extract, broccoli juice or extract, green cabbage juice or extract, onion juice or extract, garlic juice or extract, asparagus juice or extract, olive juice or extract, cucumber juice or extract, bilberry juice or extract, grapefruit juice or extract, papaya juice or extract, pineapple juice or extract, strawberry juice or extract, apple juice or extract, apricot juice or extract, cherry juice or extract, orange juice or extract, black currant juice or extract, beetroot, kiwi fruit, watermelon, aronia berry, hawthorn berry, pomegranate, fermented pomegranate, celery, cili Fruit, jujube fruit, broccoli, blue honeysuckle fruit, strawberry, yumberry, purple sweet potato, monk fruit, plum, and the like, or a combination thereof.

The antioxidant can be present in the composition in any suitable amount to provide a therapeutic effect. In one example, the antioxidant can be present in the composition in an amount of from about 0.0001 wt % to about 90 wt %. In another example, the antioxidant can be present in the composition in an amount of from about 0.0001 wt % to about 50 wt %. In another example, the antioxidant can be present in the composition in an amount of from about 0.0001 wt % to about 30 wt %. In yet another example, the antioxidant can be present in the composition in an amount of from about 0.0001 wt % to about 20 wt %.

The composition can further comprise a suitable pharmaceutically acceptable carrier. The pharmaceutically acceptable carrier can include at least one of water, a solubilizing agent or dispersing agent, a tonicity agent, a pH adjuster, a buffering agent, a preservative, a chelating agent, a bulking agent, a binder, a disintegrant, a filler, a glidant, a lubricant, a sweetener, a thickening agent, the like, and a combination thereof.

Non-limiting examples of solubilizing agents and/or emulsifiers can include water, ethanol, propylene glycol, ethylene glycol, glycerin, polyethylene glycol, banzalkonium chloride, benzethonium chloride, cetylpyridinium chloride, docusate sodium, nonoxynol-9, octoxynol, polyoxyethylene polyoxypropylene co-polymers, polyoxyl castor oils, polyoxyl hydrogenated castor oils, polyoxyl oleyl ethers, polyoxyl cetylstearyl ethers, polyoxyl stearates, polysorbates, sodium lauryl sulfate, sorbitan monolaurate, sorbitan monooleate, sorbitan monopalmitate, sorbitan monostearate, tyloxapol, the like, or combinations thereof.

In some examples, the solubilizing agent can also include a hydrocarbon or fatty substance, such as petrolatum, microcrystalline wax, paraffin wax, mineral oil, ceresi, coconut oil, bees wax, olive oil, lanolin, peanut oil, spermaceti wax, sesame oil, almond oil, hydrogenated castor oils, cotton seed oil, soybean oil, corn oil, hydrogenated sulfated castor oils, cetyl alcohol, stearyl alcohol, oleyl alcohol, lauryl alcohol, myristyl alcohol, stearic acid, oleic acid, palmitic acid, lauraic acid, ethyl oleate, isopropyl myristicate, the like, or combinations thereof. In some examples, the solubilizing agent can include a silicon, such as polydimethylsiloxanes, methicones, dimethylpropylsiloxanes, methyl phenyl polysiloxanes, steryl esters of dimethyl polysiloxanes, ethoxylated dimethicones, ethoxylated methicones, the like, or combinations thereof.

In another aspect, the pharmaceutically acceptable carrier can include a tonicity agent. Non-limiting examples of tonicity agents can include sodium chloride, potassium chloride, calcium chloride, magnesium chloride, mannitol, sorbitol, dextrose, glycerin, propylene glycol, ethanol, trehalose, phosphate-buffered saline (PBS), Dulbecco's PBS, Alsever's solution, Tris-buffered saline (TBS), water, balanced salt solutions (BSS), such as Hank's BSS, Earle's BSS, Grey's BSS, Puck's BSS, Simm's BSS, Tyrode's BSS, and BSS Plus, the like, or combinations thereof. The tonicity agent can be used to provide an appropriate tonicity of the composition. In one aspect, the tonicity of the composition can be from about 250 milliosmoles/liter (mOsm/L) to about 500 mOsm/L. In another aspect, the tonicity of the composition can be from about 250 to about 350 milliosmoles/liter (mOsm/L). In yet another aspect, the tonicity of the composition can be from about 277 to about 310 mOsm/L.

In another aspect, the pharmaceutically acceptable carrier can include a pH adjuster or buffering agent. Non-limiting examples of pH adjusters or buffering agents can include a number of acids, bases, and combinations thereof, such as hydrochloric acid, phosphoric acid, citric acid, sodium hydroxide, potassium hydroxide, calcium hydroxide, acetate buffers, citrate buffers, tartrate buffers, phosphate buffers, triethanolamine (TRIS) buffers, the like, or combinations thereof. Typically, the pH of the therapeutic composition can be from about 5 to about 9, or from about 6 to about 8. In another example, the pH of the therapeutic composition can be from about 5 to about 6. In yet another example, the parenteral composition can have a pH of from about 7 to about 8.

In another aspect, the pharmaceutically acceptable carrier can include a preservative. Non-limiting examples of preservatives can include ascorbic acid, acetylcysteine, bisulfite, metabisulfite, monothioglycerol, phenol, meta-cresol, benzyl alcohol, methyl paraben, propyl paraben, butyl paraben, benzalkonium chloride, benzethonium chloride, butylated hydroxyl toluene, myristyl gamma-picolimium chloride, 2-phenoxyethanol, phenyl mercuric nitrate, chlorobutanol, thimerosal, tocopherols, the like, or combinations thereof.

In another aspect, the pharmaceutically acceptable carrier can include a chelating agent. Non-limiting examples of chelating agents can include ethylenediaminetetra acetic acid, calcium, calcium disodium, versetamide, calteridol, diethylenetriaminepenta acetic acid, the like, or combinations thereof.

In other aspects, the pharmaceutically acceptable carrier can include a bulking agent. Non-limiting examples of bulking agents can include sucrose, lactose, trehalose, mannitol, sorbitol, glucose, rafinose, glycine, histidine, polyvinyl pyrrolidone, the like, or combinations thereof.

In other aspects, the pharmaceutically acceptable carrier can include a binder. Non-limiting examples of binders can include lactose, calcium phosphate, sucrose, corn starch, microcrystalline cellulose, gelatin, polyethylene glycol (PEG), polyvinyl pyrrolidone (PVP), hydroxypropyl cellulose, hydroxyethylcellulose, carboxymethyl cellulose (CMC), cellulose, other cellulose derivatives, the like, or combinations thereof.

In other aspects, the pharmaceutically acceptable carrier can include a thickening agent. Non-limiting examples of thickeners can include polyacrylic acids, gelatin, pectin, tragacanth, sodium alginate, methylcellulose, hydroxyethylcellulose, hydroxypropylcellulose, HPMC, CMC, alginate, starch, polyvinyl alcohol, polyvinyl pyrrolidone, co-polymers of polyoxyethylene and polyoxypropylene, polyethylene glycol, microcrystalline cellulose, tragacanth, xanthan gum, bentonite, carrageenan, guar gum, colloidal silicon dioxide, the like, or combinations thereof.

In other aspects, the pharmaceutically acceptable carrier can include: a disintegrant (e.g., crosslinked PVP, crosslinked CMC, modified starch, sodium starch glycolate); a filler (e.g., lactose, dicalcium phosphate, sucrose, or microcrystalline cellulose,) a glidant (e.g., fumed silica, talc, or magnesium carbonate); a lubricant (e.g., talc, silicon dioxide, magnesium stearate, calcium stearate, stearic acid); a sweetener (natural and/or artificial sweeteners, such as sucrose, glucose, fructose, stevia, erythritol, xylitol, aspartame, sucralose, neotame, acesulfame potassium, saccharin, advantame, sorbitol, the like, or combinations thereof).

### Dosage Forms

The nature of the pharmaceutically acceptable carrier can depend on the intended mode of administration. In some examples, the composition can be formulated for oral administration (e.g., as an oral dosage form). In other examples, the composition can be formulated for parenteral administration (e.g., via injection or IV administration).

Where the composition is formulated for oral administration, the pharmaceutically acceptable carrier can include one or more components suitable for such a composition. In the case of solid oral compositions or dosage forms, the pharmaceutically acceptable carrier can include a variety of components suitable for forming a capsule, tablet, or the like. In the case of a liquid oral composition or dosage form, the pharmaceutically acceptable carrier can include a variety of components suitable for forming a dispersion, a suspension, a syrup, an elixir, or the like.

In some specific examples, the oral dosage forms can be solid oral dosage forms. When this is the case, the solid oral dosage forms can include any pharmaceutically acceptable carrier components suitable for a solid oral dosage form. In some specific examples, the solid oral dosage form can include one or more of a binder, a disintegrant, a filler, an anti-adherent, a colorant, a glidant, a lubricant or anti-caking agent, a preservative, a desiccant, the like, or a combination thereof, such as those described above with respect to the composition. In some examples, the solid oral dosage form can be formulated as a tablet. In other examples, the solid oral dosage form can be formulated as a two-piece hard capsule or a hermetically sealed soft-gel capsule. Exterior coatings can also be used with solid oral dosage forms.

In some specific examples, the composition can be formulated as a tablet. In such examples, the composition can typically include a binder. In some examples, the composition can also include a disintegrant. In some examples, the tablet can also include a filler. In some further examples, the tablet can include an exterior coating. Such coatings can be formed with a variety of materials, such as hydroxypropyl methylcellulose (HPMC), shellac, zein, various polysaccharides, various enterics, the like, or combinations thereof. In some examples, the tablet can include a variety of other ingredients, such as anti-adherents (e.g., magnesium stearate, calcium stearate, for example), colorants (e.g., titanium dioxide, carmine, for example), glidants, lubricants or anti-caking agents, preservatives, desiccants, and/or other suitable tablet excipients, as desired.

In some other examples, the therapeutic composition can be formulated as a capsule. In such examples, the capsule itself can typically include gelatin, hypromellose, HPMC, CMC, other plant-based capsule materials, the like, or combinations thereof. A variety of excipients can also be included within the capsule, such as binders, disintegrants, fillers, glidants, anti-caking agents, preservatives, exterior coatings, the like, or combinations thereof, such as those listed above with respect to tablets, for example, or other suitable variations.

In some examples, the composition can be formulated as a liquid composition or liquid oral dosage form. A liquid oral dosage form can include a variety of excipients, such as a liquid vehicle, a solubilizing agent, a thickener or dispersant, a preservative, a tonicity agent, a pH adjuster or buffering agent, a sweetener, a thickening agent, the like, or a combination thereof. Non-limiting examples of liquid vehicles can include water, ethanol, glycerol, propylene glycol, the like, or combinations thereof.

In some examples, the composition can be formulated as a functional food and/or medical food product such as chewable gummies, a food bar, powder, or beverage. As a general guideline, the chewable gummy formulation can comprise sweeteners, gelling agents, acidulants, colorants, and flavoring agents. One design objective is to achieve the proper texture, sweetness, flavor release, and stability. Some gummies may be tougher and have the tendency to cleave on chewing while others tend to be softer and chewier. Food bars can be formulated to fit different dietary regiments for any specific purposes such as weight loss, energy, meal replacement, high protein, high fiber, low glycemic, etc. A food bar usually contains ingredients that supply energy-yielding nutrients such as carbohydrate, protein and lipid as well as other macro- and micronutrients including but not limited to vitamins and minerals. Other health promoting ingredients such fruit and vegetable powder may be included in the formulation in addition to filler, binder, emulsifier, water, humectant, flavor, color, sweetener, preservative, etc. The composition can be formulated into a food bar with other ingredients to achieve desirable health benefits, taste, texture, flavor and stability. Similarly, the composition may be formulated into a powder such as a protein powder, meal replacement powder, or functional beverage dry mix. It can also be formulated into a functional drink. A ready to drink beverage may contain other ingredients including various nutrients, health promoting agents, pH adjustor (acidity regulator), electrolyte, flavor, sweetener, stabilizing agent, color, preservative, etc.

In yet additional alternatives, the therapeutic compositions described herein can be used as a food additive to fortify a food supply for general population. For example, the therapeutic composition can be safely introduced into a systemic food supply such as, but not limited to, milled grain flours, pastas, breakfast cereals, bread, soup or soup mixes, food bars, spices, condiments, dairy products, beverages, drink mixes, frozen food items, pastries, cookies and crackers, snacks, or the like.

In some examples, the oral dosage form can include COS in an amount from about 0.5 mg to about 5,000 mg per dose. In some other examples, the oral dosage form can include at least one of COS in an amount of from about 5 mg to about 1500 mg per dose. In some additional examples, the oral dosage form can include at least one of COS with at least one of hMO and rhamnan sulfate in an amount of from about 5 mg to about 500 mg per dose. In still other examples, the oral dosage form can include at least one of COS with at least one of hMO and rhamnan sulfate in an amount of from about 50 mg to about 1000 mg per dose, and in some cases about 5 mg to about 100 mg per dose.

When the composition is formulated for administration via injection, the pharmaceutically acceptable carrier can include one or more components suitable for such a composition. Non-limiting examples can include water, a solubilizing or dispersing agent, a tonicity agent, a pH adjuster or buffering agent, a preservative, a chelating agent, a bulking agent, the like, or a combination thereof.

In some examples, the dosage forms or compositions described herein can be disposed in a suitable container. Such containers can include multiple-use containers or single use containers. Non-limiting examples can include bottles, vials, bags, or the like. In some examples, the container can be an amber colored container or other suitable container configured to protect the dosage form or therapeutic composition from light. In yet other examples, the container can include instructions and dosing information for the dosage form or therapeutic composition. The container can include a variety of materials, such as polyethylene, polypropylene, polycarbonate, polyvinyl chloride, glass, the like, or a combination thereof.

### Methods of Treatment

The present disclosure also describes a method of treating intestinal glycocalyx dysfunction in a subject. Intestinal glycocalyx dysfunction can include any dysfunction caused by a disorder in the epithelial glycocalyx in the gastrointestinal tract. A disorder in the intestinal epithelial glycocalyx can include disorders arising from damage in the epithelial glycocalyx, such as reduced intestinal glycocalyx thickness, reduced intestinal glycocalyx length, reduced intestinal glycocalyx volume, reduced intestinal glycocalyx integrity, the like, and a combination thereof. In some examples, intestinal glycocalyx dysfunction can comprise at least one of inflammatory bowel diseases, irritable bowel syndrome, celiac disease, small intestinal bacterial overgrowth (SIBO), leaky gut syndrome, colon cancer, the like, and a combination thereof.

The method of treating intestinal glycocalyx dysfunction can comprise identifying intestinal glycocalyx dysfunction in a subject. Intestinal glycocalyx dysfunction can be detected using any appropriate method such as biomarkers, imaging technologies, and functional tests. In one example, at least one of biomarkers (i.e., plasma Zonulin zonulin antibodies, occluding, and lipopolysaccharide), lectin staining and light/electron microscopy of intestinal tissue biopsies, intestinal permeability assay, confocal laser endomicroscopy, the like, and a combination thereof can be used to identify intestinal glycocalyx dysfunction in a subject.

A composition can be administered to the subject in an amount and at a frequency sufficient to stabilize and reverse damage in the intestinal glycocalyx, as well as to maintain at least one of an intestinal glycocalyx thickness, an intestinal glycocalyx length, an intestinal glycocalyx volume, an intestinal glycocalyx integrity, an intestinal glycocalyx function, and a combination thereof. The composition can comprise any suitable composition as disclosed herein. In one example, the composition can comprise a COS in an amount and at a frequency sufficient to prevent, stabilize, and reverse damage in an intestinal glycocalyx. In one example, the composition can comprise a combination of a rhamnan sulfate and a COS. In another example, the composition can comprise a combination of a COS and an hMO. In another example, the composition can comprise a combination of rhamnan sulfate, a COS, and an hMO.

As otherwise disclosed herein, the COS can have a degree of polymerization of less than or equal to 50 and an average molecular weight of less than 10 kilodaltons. In another example, the rhamnan sulfate can have an average molecular weight (MW) of less than 2000 kDa. In yet another example, the hMO can be at least one of a neutral hMO, a neutral N-containing hMO, an acid hMO, the like, and a combination thereof.

As otherwise disclosed herein, in one example, the rhamnan sulfate can have an average molecular weight (MW) of less than 2000 kDa. In another example, the COS can be a chitosan having a polymerization of less than or equal to 50 and an average molecular weight of less than 10 kDa. In yet another example, the hMO can be at least one of a neutral hMO, a neutral N-containing hMO, an acid hMO, the like, and a combination thereof.

The COS, hMO, and rhamnan sulfate can be administered in any suitable amount and at any suitable frequency. In one example, when present, the rhamnan sulfate can be administered in an amount of from about 50 mg to about 1000 mg per dose at a frequency of 1 to 5 times daily. In one example, the COS can be administered to the subject in an amount of from about 50 mg to about 2500 mg per dose at a frequency of 1 to 5 times daily. In another example, the hMO can be administered to the subject in an amount of from about 10 mg to about 5000 mg per dose at a frequency of 1 to 5 times daily. In another example, the rhamnan sulfate can be administered in an amount of from about 10 mg to about 1000 mg per dose at a frequency of 1 to 5 times daily.

An intestinal glycocalyx precursor can also be administered to the subject. The method can further comprise administering an intestinal glycocalyx precursor to the subject. The intestinal glycocalyx precursor can be a glycan comprising sialic acid, glucosamine, hyaluronic acid, chondroitin sulfate, heparan sulfate, dermatan sulfate, the like, or a combination thereof as otherwise disclosed herein. The intestinal glycocalyx precursor can be administered to the subject in an amount of from about 10 mg to about 5000 mg per dose at a frequency of 1 to 5 times daily.

In a further example, an antioxidant and/or polyphenol can be administered to the subject. The antioxidant and/or polyphenol can be administered to the subject in an amount of from about 50 mg to about 5000 mg per dose at a frequency of 1 to 5 times daily.

Administration of the composition (e.g., of at least one of a COS, an hMO, and a rhamnan sulfate) to a subject can be performed in a number of ways. In some examples, the composition can be administered orally. Oral administration can include administration as a solid oral dosage form (e.g., a tablet including chewable and lozenge, a capsule, etc.) or a liquid oral dosage form (e.g., a solution, a suspension, a syrup, an elixir, a gel, etc.). In some other examples, administration can be performed via injection (e.g., intravenous, intra-arterial, intramuscular, sub-cutaneous, etc.). Further, where the composition is administered via injection, it can be injected via a bolus injection or via metered infusion. Other forms of administration can also include sublingual/buccal mucosal delivery, topical administration, transdermal administration, inhalation, ophthalmic administration, nasal administration, otic administration, administration as a suppository, or the like.

The particular composition administered can be any of those described herein, or the like. Further, in some examples, the composition (e.g., of at least one of a COS, an hMO, and a rhamnan sulfate) can be administered as a composition or dosage form, such as those described herein. In some examples, the oral dosage form can be administered in an amount from about 50 mg per dose to about 500 mg per dose. In some other examples, the oral dosage form can be administered in an amount from about 500 mg to about 2000 mg per dose. In some additional examples, the oral dosage form can be administered in an amount from about 250 mg to about 2500 mg per dose. In still other examples, the oral dosage form can be administered in an amount from about 500 mg to about 5000 mg per dose. It is also noted that where the combination of the at least one of a COS, an hMO, and a rhamnan sulfate is administered as part of a solid oral dosage form, a dose can include one, two, three, four, or more capsules, tablets, etc.

The combination of at least one of a COS, an hMO, and a rhamnan sulfate can be administered at a variety of frequencies. In some examples, a dose of the combination of at least one of a COS, an hMO, and a rhamnan sulfate can be administered at a frequency of from once daily to four times daily. In some examples, a dose of the combination of at least one of a COS, an hMO, and a rhamnan sulfate can be administered once per day, twice per day, three times per day, four times per day, or more. In other examples, the combination of at least one of a COS, an hMO, and a rhamnan sulfate can be administered at a frequency of from about once every two days, three days, five days, or seven days, for example. Thus, a variety of suitable administration frequencies can be employed with the present methods.

Further, administration can continue for a variety of durations, depending on the desired treatment outcome. In some examples, administration can continue while symptoms of intestinal glycocalyx dysfunction persist. In other examples, administration can be ongoing as either a prophylactic or intervention treatment. In still other examples, administration can continue until the intestinal glycocalyx dysfunction has resolved or a threshold level of intestinal glycocalyx stabilization or reversal in damage has been reached. Other suitable durations of administration can also be employed, as desired. As a general guideline, administration duration can be from about 2 weeks to about 24 months, and often from 2 months to 12 months.

In some examples, the combination at least one of a COS, an hMO, and a rhamnan sulfate can be administered in connection (e.g., co-administered) with another active agent. In some examples, the second active agent can include a glycocalyx precursor, antioxidant, polyphenol, any other suitable active agent, or a combination thereof, as described elsewhere herein.

### EXAMPLES

The following examples are provided to promote a clearer understanding of certain embodiments of the present disclosure and are in no way meant as a limitation thereon.

### Example 1: COS Repairs the Damage of Glycocalyx Damage of Caco-2 Intestinal Epithelial Cells Caused by Dextrin Sulfate Sodium (DSS)

Caco-2 cells were grown to 90% confluency in a 24-well cell culture plate. 2% DSS was added into each well for another 24 hours. Cell culture medium in each well was then removed. Fresh new medium was added to control wells while fresh new medium with 200 μg/ml COS was added to the test wells. Four different COS' were tested for another 24 hours. At the end of the experiments, Caco-2 cells were stained with wheat germ agglutinin fluorescein isothiocyanate (WGA-FITC) and 4′,6-diamidino-2-phenylindole (DAPI) for the glycocalyx and nuclear DNA. Florescence intensity was measured and analyzed with ImageJ software. The degree of polymerization of 4 COS' ranges from 2 to 40 with a degree of deacetylation of 10-95%. Table I below lists the degree of polymerization (DP) of each COS used in the experiments. Table II shows the results of average florescence intensity of the Caco-2 glycocalyx after WGA-FITC staining. Incubation of Caco-2 cells with 2% DSS for 24 hours causes a 52.38% decrease of their glycocalyx as measured by florescence intensity at the end of 72 hours of incubation. When 200 μg/ml COS was added to cell culture media for the last 24 hours, florescence intensity increased dramatically compared to the control without any COS. COS A is the most effective and brought the florescence intensity back to 76.75% of the control. COS B, C, D had a similar effect on the florescence intensity at about 80% of the control. These data clearly demonstrate that COS can repair, regenerate, and restore the intestinal epithelial glycocalyx. Because COS A works well to protect and restore the intestinal glycocalyx in this study, it is chosen to be used in the animal study below.

TABLE I
(Degree of Polymerization of COS’)

| COS | DP |
|---|---|
| A | 2-15 |
| B | 5-35 |
| C | 2-25 |
| D | 2-10 |

TABLE II
(Average Florescence Intensity after WGA-FITC Staining)

| Sample | Average Florescence Intensity (Au) |
|---|---|
| Control | 118.80 |
| DSS Treated | 56.87 |
| COS A | 91.18 |
| COS B | 78.70 |
| COS C | 83.81 |
| COS D | 77.73 |

### Example 2: RS, COS and hMO Prevent and Repair the Damage of Glycocalyx Damage of Caco-2 Intestinal Epithelial Cells Caused by Dextrin Sulfate Sodium (DSS)

Using the same microfluidic chip model in Example 1, different functional glycans of RS, COS and hMO, and some combinations were evaluated for their capability to prevent and repair the damages of Caco-2 intestinal epithelial cell glycocalyx caused by DSS. Table III shows the different treatments of Caco-2 epithelial cells in the experiments. In addition to DSS, a positive control of 5-aminosalicylic acid (5-ASA), RS, COS, different hMOS, and a combination of hMOs and COS at different concentrations in phosphate-buffered saline (PBS) with water were introduced into microfluidic chips for testing their effects on the intestinal glycocalyx in the presence of DSS.

TABLE III
(Experimental Design)

| Group | PBS/H₂O | DSS | 5-ASA | RS | COS | 6′-SL | 3′-SL | 2′-FL |
|---|---|---|---|---|---|---|---|---|
| Control | ✓ | / | / | / | / | / | / | / |
| Negative | / | 2% | | / | 1 | / | / | 1 |
| Positive | 1 | 2% | 200 μg/ml | / | / | / | / | 1 |
| RS | / | 2% | / | 200 μg/ml | 1 | / | / | / |
| COS | / | 2% | / | / | 200 μg/ml | / | / | / |
| 6′-SL | / | 2% | / | / | / | 200 1 μg/ml | / | / |
| 6′-SL + 3′-SL | / | 2% | / | 1 | / | 100 1 μg/ml | 100 μg/ml | / |
| HMOs + COS | 1 | 2% | / | / | 100 μg/ml | 50 μg/ml | 50 μg/ml | 50 μg/ml |

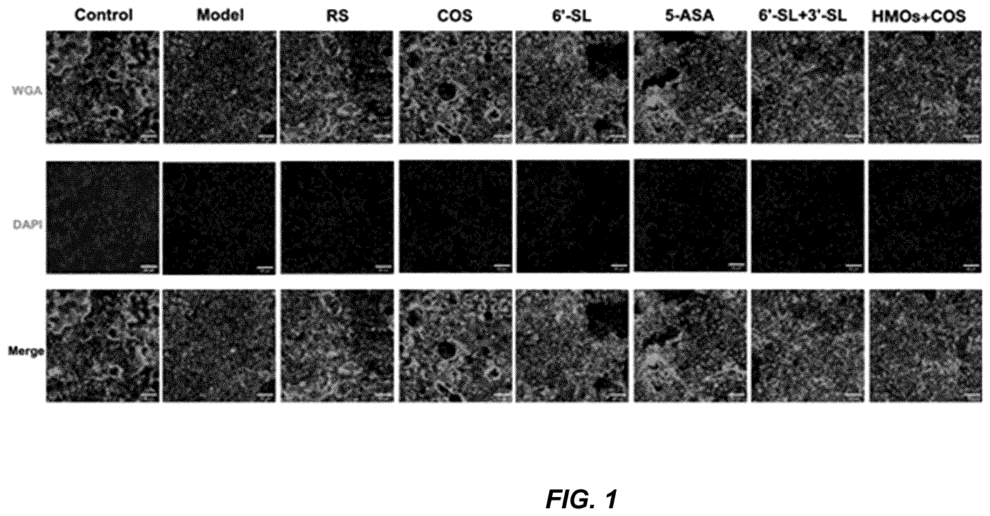

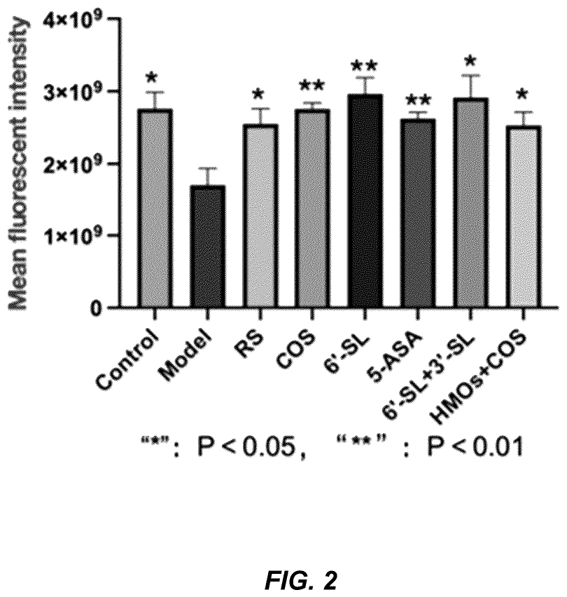

The experimental results were shown in FIG. 1 and FIG. 2 below. FIG. 1 depicts the images of the intestinal epithelial cell glycocalyx and nucleus stained by WGA-FITC individually and together. FIG. 2 shows the average fluorescence intensity of the staining in a bar chart. Briefly, 2% DSS caused a significant decrease of intestinal glycocalyx as measured by WGA fluorescence intensity. All treatment including RS, 5-ASA, COS, 6′-SL each individually at 200 μg/ml, 6′-SL and 3′-SL each at 100 μg/ml together, and a combination of 100 μg/n COS with 50 μg/ml each of 6′-SL, 3′-SL and 2′-FL significantly prevented the damage of the intestinal glycocalyx caused by DSS.

### Example 3: RS, COS and hMOs Individually and in Combinations Protect Intestinal Glycocalyx Against Damages Caused by Dextrin Sulfate Sodium (DSS) in Mice

Male C57BL/6J mice (18-20 g) of 6 weeks old were housed under controlled conditions with a constant temperature (23±2° C.), stable humidity, and a 12-hour light/dark cycle. After a one-week acclimatization period, the mice were randomly divided into 7 groups, with 5 mice per group: Control, DSS, DSS+3′-SL, DSS+6′-SL, DSS+5-ASA, DSS+6′-SL+3′-SL, and DSS+HMOs+COS. The control group received sterile water for 19 days. The DSS group was given 4% (wt/vol) DSS in their drinking water from days 8-14, with daily oral gavage of sterile water for 11 days. The other groups were treated with 3% DSS in their drinking water from days 8-14, along with oral administration of various functional glycans (mg/kg/day) of COS up to the 19th day (See Experimental Design in Table III). The DSS solution was replaced every two days to maintain its bioactivity. Mouse body weight, stool consistency, and rectal bleeding were monitored daily, alongside observations of coat sheen, mental state, and activity levels.

The Disease Activity Index (DAI) was used to assess colitis severity, calculated as the mean of the scores for weight loss, stool consistency, and rectal bleeding as described below. Mice were weighed daily at the same time, with the percentage of weight loss compared to the end of the acclimation period: <1% scored 0; 1-5% scored 1; 5-10% scored 2; 10-20% scored 3; >20% scored 4. Stool consistency was also evaluated daily: normal stool scored 0; soft but formed stool scored 1; paste-like stool scored 2; liquid stool scored 3. Rectal bleeding was assessed using a fecal occult blood test kit: negative scored 0; positive without visible blood scored 1; positive with visible blood in stool scored 2; visible blood on the anus scored 3. The DAI was calculated as the average of these three scores. All measurements for DAI started from day 8, when DSS was introduced.

Hematoxylin-Eosin (HE) staining was performed using the following procedure. After 19 days of functional glycan intervention, mice were euthanized, and distal colon tissues were collected. The tissues were fixed in 4% paraformaldehyde for 24 hours and then processed for HE staining. The fixed tissues were dehydrated through a graded ethanol series, cleared in xylene, embedded in paraffin, and sectioned into 5 m slices. Deparaffinization was performed with xylene (5 min×2). The sections were rehydrated in descending concentrations of ethanol and stained with hematoxylin for 5 minutes, followed by eosin for 2 minutes. After dehydration, the sections were cleared in xylene (5 min×2), mounted with neutral resin, and examined under a microscope for morphological analysis. Nuclei stained blue, and cytoplasm stained red.

Immunofluorescence staining of mouse colon tissue was performed using the following procedure. Paraffin sections were prepared from the distal colon, 1 cm from the anus. Deparaffinization and rehydration were performed with xylene and ethanol, as follows: xylene (5 min×2), 100% ethanol (5 min×2), 90% ethanol (5 min×1), 80% ethanol (5 min×1), 70% ethanol (5 min×1), and deionized water (10 s). Endogenous peroxidase activity was quenched with 0.75% hydrogen peroxide for 10 minutes, followed by PBS washes. Antigen retrieval was performed by boiling the sections in 0.01 mol/L sodium citrate for 30 minutes. For WGA staining, sections were incubated overnight at 4° C. with 0.1 μg/μL FITC-WGA in PBS (40 μL per sample). After washing with PBS, nuclei were counterstained with 30 DAPI and sections were mounted with an anti-fade medium. The stained sections were visualized under a fluorescence microscope, and the colonic mucosal glycocalyx was quantified using ImageJ software.

TABLE IV
(Experimental Design)

| Group | H2O | 3% DSS | 3′-SL | 6′-SL | 2′-FL | COS | 5-ASA |
|---|---|---|---|---|---|---|---|
| Control | ✓ | / | / | / | / | / | / |
| Model | / | ✓ | / | / | / | / | / |
| 3′-SL | / | ✓ | 100 | / | / | / | / |
| 6′-SL | / | ✓ | / | 100 | / | / | / |
| 5-ASA | / | ✓ | / | / | / | / | 100 |
| 3′-SL + 6′-SL | / | ✓ | 100 | 100 | / | / | / |
| HMOs + COS | / | ✓ | 50 | 50 | 50 | 100 | / |

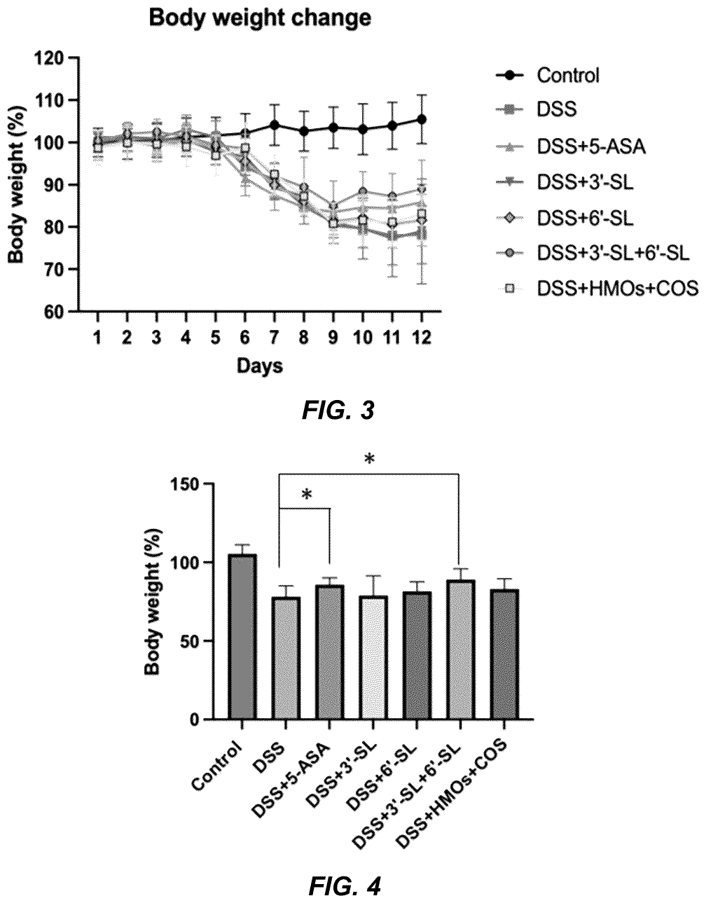

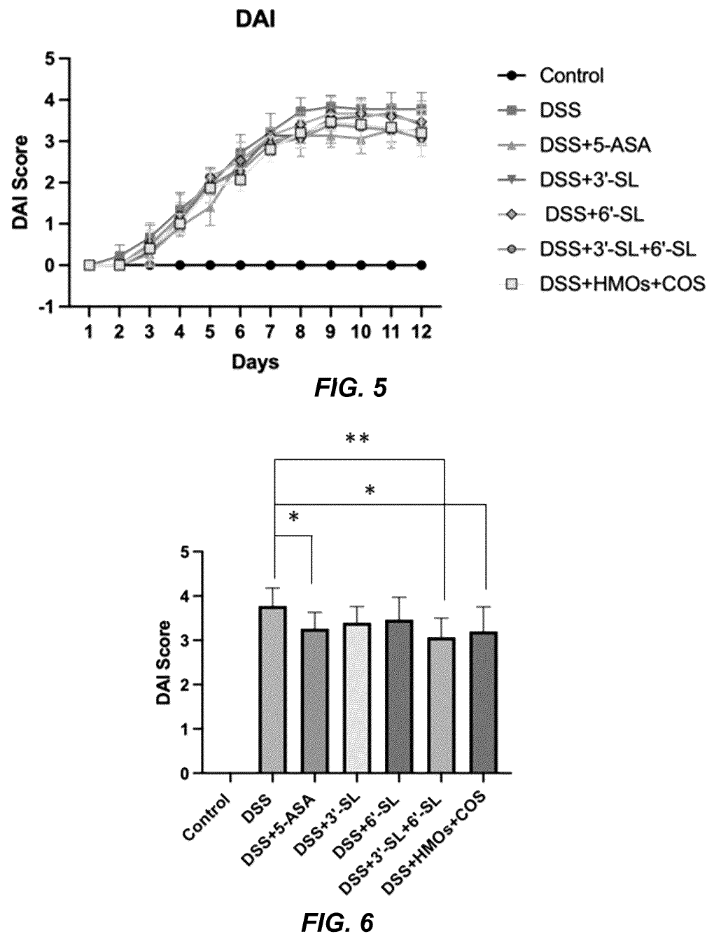

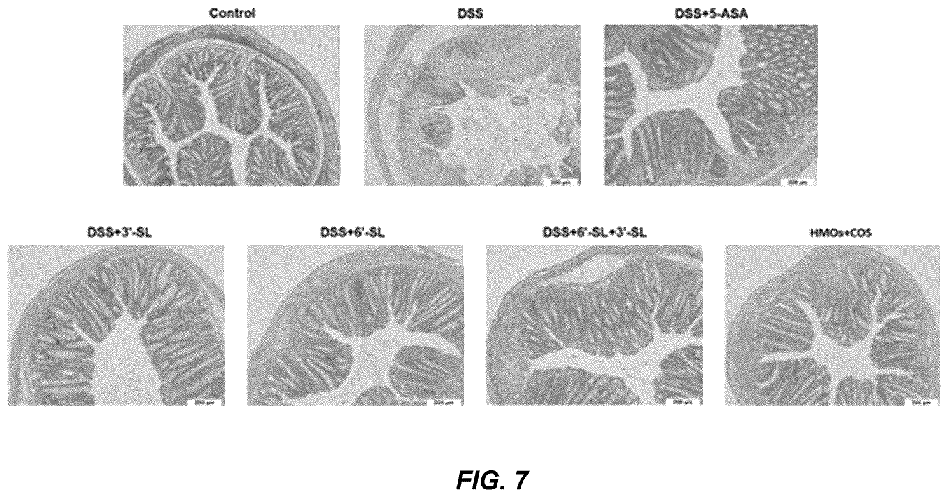

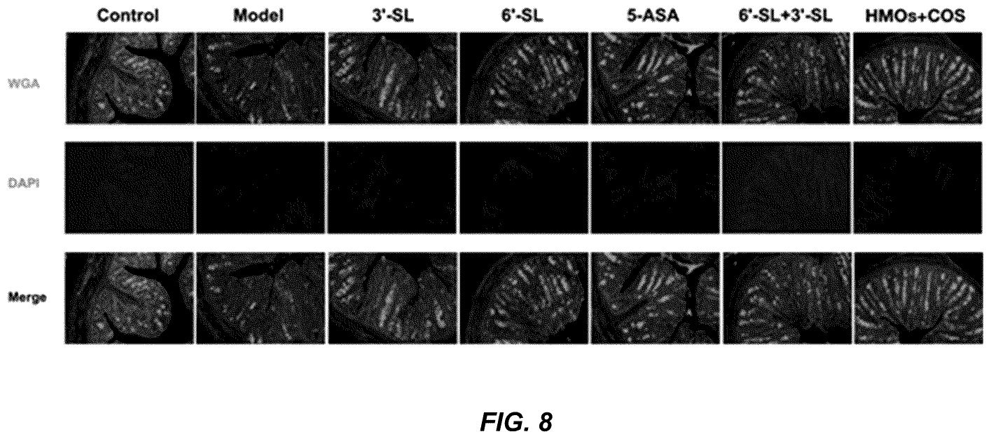

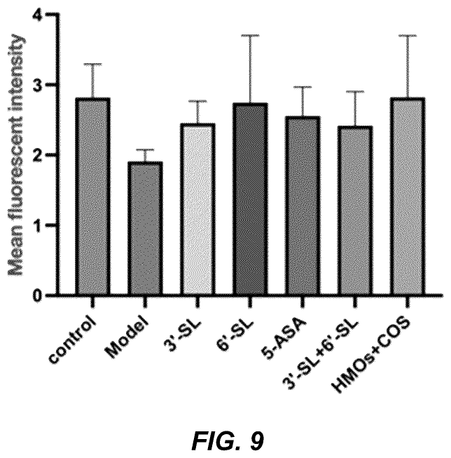

Treatment of DSS caused significant body weight reduction and DAI score. Each of the interventions showed a positive impact on body weight and DAI (FIGS. 3-7) in the 12 days following the introduction of DDS to drinking water. At the end of the study, mice treated with a combination of 3′-SL and 6′-SL had a significant increase in their body weight compared to the mice treated with DSS only in addition to the positive control (FIG. 4). At the same time, mice treated with the same combination of 3′-SL and 6′-SL and also a combination of 3′-SL, 6′-SL, 2′-FL and HMO significantly reduced the DAI compared to the DSS treatment only in addition to the positive control (FIG. 6). Histological analysis after HE staining shows that DDS caused significant tissue damage in the mouse colon. Every intervention of hMOs and the combination of hMOs and COS showed protection again the damage (FIG. 6). Most importantly, DSS significantly reduced the mouse intestinal glycocalyx (FIG. 8 and FIG. 9). FIG. 8 contains the images of the mouse intestinal epithelial cell glycocalyx stained with WGA. FIG. 9 shows the average fluorescence intensity of the staining in a bar chart. All intervention groups including 3′-SL, 6′-SL, 3′-SL with the combination of 6′-SL, 3′-SL, 6′-SL, 2′-FL and COS increased the mouse intestinal glycocalyx (Table V).

TABLE V
(Average Florescence Intensity of Mouse Intestinal
Glycocalyx after WGA-FITC Staining)

| Group | Control | Model | 3′-SL | 6′-SL | 5-ASA | 3′-SL + 6′SL | HMOs + COS |
|---|---|---|---|---|---|---|---|
| AVI | 2.89 | 1.91 | 2.45 | 2.75 | 2.56 | 2.42 | 2.82 |
| Std Dev | 0.52 | 0.17 | 0.31 | 0.96 | 0.41 | 0.49 | 0.88 |

Based on the data from Examples 1-3, below compositions are prepared to protect and enhance structural and functional integrity of the intestinal glycocalyx in humans. According to the FDA guidelines and other scientific references, dose conversion from animal to human should be based primarily on the body surface area (BSA) since BSA has good correlation among species with several parameters including oxygen utilization, caloric expenditure, basal metabolism, blood volume and circulating plasma proteins. Specifically, for mouse, a factor of 10 may be used for the convention of dose to human. In other words, doses at 50 mg/kg/day, 100 mg/kg/day and 200 mg/kg/day used in Example III for mice can be converted to 5 mg/kg/day, 10 mg/kg/day and 20 mg/kg/day for humans. For a 70 kg human being, 5 mg/kg/day, 10 mg/kg/day and 20 mg/kg/day translate to 350 mg/day, 700 mg/day and 1,400 mg/day.

### Example 4: COS Compositions

Compositions are prepared using the components set forth in Table VI-IX.

TABLE VI
(Formula 1)

| | | Wt % | Amount |
|---|---|---|---|
| Type | Ingredient | (w/w) | (mg/serving) |
| Chito-oligosaccharide | COS | 1%-100% | 30 mg-3000 mg |

TABLE VII
(Formula 2)

| | | Wt % | Amount |
|---|---|---|---|
| Type | Ingredient | (w/w) | (mg/serving) |
| Chito-oligosaccharide | COS | 1%-80% | 30 mg-2400 mg |
| Antioxidant/Polyphenol | Pomegranate | 1%-80% | 30 mg-2400 mg |
| | Extract | | |

TABLE VIII
(Formula 3)

| | | Wt % | Amount |
|---|---|---|---|
| Type | Ingredient | (w/w) | (mg/serving) |
| Chito-oligosaccharide | COS | 1%-80% | 30 mg-2400 mg |
| Glycocalyx Precursor | Sialic Acid | 1%-80% | 30 mg-2400 mg |

TABLE IX
(Formula 4)

| | | Wt % | Amount |
|---|---|---|---|
| Type | Ingredient | (w/w) | (mg/serving |
| Chito-oligosaccharide | COS | 1%-60% | 30 mg-1800 mg |
| Antioxidant/Polyphenol | Pomegranate | 1%-60% | 30 mg-1800 mg |
| | extract | | |
| Glycocalyx Precursor | Sialic Acid | 1%-60% | 300 mg-1800 mg |

### Example 5: Chito-Oligosaccharide (COS) and hMO Compositions

Compositions are prepared using the components set forth in Table X-XIII.

TABLE X
(Formula 5)

| | | Wt % | Amount |
|---|---|---|---|
| Type | Ingredient | (w/w) | (mg/serving) |
| Chito-oligosaccharide | COS | 1%-80% | 30 mg-2400 mg |
| Human milk | 3′-SL or 6′-SL | 1%-80% | 30 mg-2400 mg |
| oligosaccharide | or 2′-FL | | |

TABLE XI
(Formula 6)

| | | Wt % | Amount |
|---|---|---|---|
| Type | Ingredient | (w/w) | (mg/serving) |
| Chito-oligosaccharide | COS | 1%-60% | 100 mg-1800 mg |
| Human milk | 3′-SL or 6′-SL | 1%-60% | 30 mg-1800 mg |
| oligosaccharide | or 2′-FL | | |
| Antioxidant/ | Pomegranate | 1%-60% | 30 mg-1800 mg |
| Polyphenol | Extract | | |

TABLE XII
(Formula 7)

| | | Wt % | Amount |
|---|---|---|---|
| Type | Ingredient | (w/w) | (mg/serving) |
| Chito-oligosaccharide | COS | 1%-60% | 30 mg-800 mg |
| Human milk | 3′-SL or 6′-SL | 1%-60% | 30 mg-1800 mg |
| oligosaccharide | or 2′-FL | | |
| Glycocalyx Precursor | Sialic Acid | 1%-60% | 30 mg-1800 mg |

TABLE XIII
(Formula 8)

| | | | Amount |
|---|---|---|---|
| Type | Ingredient | Wt % (w/w) | (mg/serving) |
| Chito- | COS | 0.5%-40% | 15 mg-1200 mg |
| oligosaccharide | | | |
| Human milk | 3′-SL or 6′_SL | 0.5%-40% | 15 mg-1200 mg |
| oligosaccharide | or 2′-FL | | |
| Antioxidant/ | Pomegranate | 0.5%-40% | 15 mg-1200 mg |
| Polyphenol | Extract | | |
| Glycocalyx | Sialic Acid | 0.5%-40% | 15 mg-1200 mg |
| Precursor | | | |

### Example 6: Sulfated Polysaccharide Compositions

Compositions are prepared using the components set forth in Table XIV-XVII.

TABLE XIV
(Formula 9)

| | | | Amount |
|---|---|---|---|
| Type | Ingredient | Wt % (w/w) | (mg/serving) |
| Sulfated | Rhamnan sulfate | 1%-80% | 15 mg-1200 mg |
| polysaccharide | | | |

TABLE XV
(Formula 10)

| | | | Amount |
|---|---|---|---|
| Type | Ingredient | Wt % (w/w) | (mg/serving) |
| Sulfated | Rhamnan sulfate | 1%-80% | 15 mg-1200 mg |
| polysaccharide | | | |
| Antioxidant/ | Pomegranate | 1%-80% | 15 mg-1200 mg |
| Polyphenol | Extract | | |

TABLE XVI
(Formula 11)

| | | | Amount |
|---|---|---|---|
| Type | Ingredient | Wt % (w/w) | (mg/serving) |
| Sulfated | Rhamnan sulfate | 1%-80% | 15 mg-1,200 mg |
| polysaccharide | | | |
| Glycocalyx | Sialic Acid | 1%-80% | 15 mg-1,200 mg |
| Precursor | | | |

TABLE XVII
(Formula 12)

| | | | Amount |
|---|---|---|---|
| Type | Ingredient | Wt % (w/w) | (mg/serving) |
| Sulfated | Rhamnan sulfate | 1%-60% | 15 mg-900 mg |
| polysaccharide | | | |
| Antioxidant/ | Pomegranate | 1%-60% | 15 mg-900 mg |
| Polyphenol | Extract | | |
| Glycocalyx | Sialic Acid | 1%-60% | 15 mg-900 mg |
| Precursor | | | |

### Example 7: Sulfated Polysaccharide and Chito-Oligosaccharide (COS) Compositions

Compositions are prepared using the components set forth in Table XVIII-XXI.

TABLE XVIII
(Formula 13)

| | | | Amount |
|---|---|---|---|
| Type | Ingredient | Wt % (w/w) | (mg/serving) |
| Sulfated | Rhamnan sulfate | 1%-80% | 15 mg-1,200 mg |
| polysaccharide | | | |
| Chito- | COS | 1%-80% | 15 mg-1,200 mg |
| oligosaccharide | | | |

TABLE XIX
(Formula 14)

| | | | Amount |
|---|---|---|---|
| Type | Ingredient | Wt % (w/w) | (mg/serving) |
| Sulfated | Rhamnan sulfate | 1%-60% | 15 mg-900 mg |
| polysaccharide | | | |
| Chito-oligosaccharide | COS | 1%-60% | 15 mg-900 mg |
| Antioxidant/ | Pomegranate | 1%-60% | 15 mg-900 mg |
| Polyphenol | Extract | | |

TABLE XX
(Formula 15)

| | | | Amount |
|---|---|---|---|
| Type | Ingredient | Wt % (w/w) | (mg/serving) |
| Sulfated | Rhamnan sulfate | 1%-60% | 15 mg-900 mg |
| polysaccharide | | | |
| Chito- | COS | 1%-60% | 15 mg-900 mg |
| oligosaccharide | | | |
| Glycocalyx | Sialic Acid | 1%-60% | 15 mg-900 mg |
| Precursor | | | |

TABLE XXI
(Formula 16)

| | | | Amount |
|---|---|---|---|
| Type | Ingredient | Wt % (w/w) | (mg/serving) |
| Sulfated | Rhamnan sulfate | 1%-60% | 15 mg-900 mg |
| polysaccharide | | | |
| Chito- | COS | 1%-60% | 15 mg-900 mg |
| oligosaccharide | | | |
| Antioxidant/ | Pomegranate | 1%-60% | 15 mg-900 mg |
| Polyphenol | extract | | |
| Glycocalyx | Sialic Acid | 1%-60% | 15 mg-900 mg |
| Precursor | | | |

### Example 8: Sulfated Polysaccharide and Human Milk Oligosaccharide (hMO) Compositions

Compositions are prepared using the components set forth in Table XXII-XXV.

TABLE XXII
(Formula 17)

| | | | Amount |
|---|---|---|---|
| Type | Ingredient | Wt % (w/w) | (mg/serving) |
| Sulfated | Rhamnan sulfate | 1%-60% | 15 mg-900 mg |
| polysaccharide | | | |
| Human milk | 3′-SL or 6′-SL | 1%-60% | 15 mg-900 mg |
| oligosaccharide | or 2′-FL | | |

TABLE XXIII
(Formula 18)

| | | | Amount |
|---|---|---|---|
| Type | Ingredient | Wt % (w/w) | (mg/serving) |
| Sulfated | Rhamnan sulfate | 1%-60% | 15 mg-900 mg |
| polysaccharide | | | |
| Human milk | 3′-SL or 6′-SL | 1%-60% | 15 mg-900 mg |
| oligosaccharide | or 2′-FL | | |
| Antioxidant/ | Pomegranate | 1%-60% | 15 mg-900 mg |
| Polyphenol | extract | | |

TABLE XXIV
(Formula 19)

| | | | Amount |
|---|---|---|---|
| Type | Ingredient | Wt % (w/w) | (mg/serving) |
| Sulfated | Rhamnan sulfate | 1%-60% | 15 mg-900 mg |
| polysaccharide | | | |
| Human milk | 3′-SL or 6′-SL | 1%-60% | 15 mg-900 mg |
| oligosaccharide | or 2′-FL | | |
| Glycocalyx | Sialic Acid | 1%-60% | 15 mg-900 mg |
| Precursor | | | |

TABLE XXV
(Formula 20)

| | | | Amount |
|---|---|---|---|
| Type | Ingredient | Wt % (w/w) | (mg/serving) |
| Sulfated | Rhamnan sulfate | 1%-60% | 15 mg-900 mg |
| polysaccharide | | | |
| Human milk | 3′-SL or 6′-SL | 1%-60% | 15 mg-900 mg |
| oligosaccharide | or 2′-FL | | |
| Antioxidant/ | Pomegranate | 1%-60% | 15 mg-900 mg |
| Polyphenol | extract | | |
| Glycocalyx | Sialic Acid | 1%-60% | 15 mg-900 mg |
| Precursor | | | |

### Example 9: Sulfated Polysaccharide. COS, and hMO Compositions

Compositions are prepared using the components set forth in Table XXVI-XXIX.

TABLE XXVI
(Formula 21)

| | | | Amount |
|---|---|---|---|
| Type | Ingredient | Wt % (w/w) | (mg/serving) |
| Sulfated | Rhamnan | 1%-60% | 15 mg-900 mg |
| polysaccharide | sulfate | | |
| Chito- | COS | 1%-60% | 15 mg-900 mg |
| oligosaccharide | | | |
| Human milk | 3′-SL or 6′-SL | 1%-60% | 15 mg-900 mg |
| oligosaccharide | or 2′-FL | | |

TABLE XXVII
(Formula 22)

| | | | Amount |
|---|---|---|---|
| Type | Ingredient | Wt % (w/w) | (mg/serving) |
| Sulfated | Rhamnan | 1%-60% | 15 mg-900 mg |
| polysaccharide | sulfate | | |
| Chito- | COS | 1%-80% | 15 mg-1,200 mg |
| oligosaccharide | | | |
| Human milk | 3′-SL or 6′-SL | 1%-80% | 15 mg-1200 mg |
| oligosaccharide | or 2′-FL | | |
| Antioxidant/ | Pomegranate | 1%-80% | 15 mg-1200 mg |
| Polyphenol | extract | | |

TABLE XXVIII
(Formula 23)

| | | Wt % | Amount |
|---|---|---|---|
| Type | Ingredient | (w/w) | (mg/serving) |
| Sulfated | Rhamnan | 1%-40% | 15 mg-600 mg |
| polysaccharide | sulfate | | |
| Chito-oligosaccharide | COS | 1%-80% | 15 mg-1200 mg |
| Human milk | 3′-SL or 6′-SL | 1%-60% | 15 mg-900 mg |
| oligosaccharide | or 2′-FL | | |
| Glycocalyx Precursor | Sialic Acid | 1%-60% | 15 mg-900 mg |

TABLE XXIX
(Formula 24)

| | | Wt % | Amount |
|---|---|---|---|
| Type | Ingredient | (w/w) | (mg/serving) |
| Sulfated polysaccharide | Rhamnan sulfate | 1%-40% | 15 mg-600 mg |
| Chito-oligosaccharide | COS | 1%-80% | 15 mg-1200 mg |
| Human milk | 3′-SL or 6′-SL or | 1%-60% | 15 mg-900 mg |
| oligosaccharide | 2′-FL | | |
| Antioxidant/Polyphenol | Pomegranate extract | 1%-60% | 15 mg-900 mg |
| Glycocalyx Precursor | Sialic Acid | 1%-60% | 15 mg-900 mg |

### Example 10: Human Milk Oligosaccharide (hMO) Compositions

Compositions are prepared using the components set forth in Table XXX-XXXV.

TABLE XXX
(Formula 25)

| | | Wt % | Amount |
|---|---|---|---|
| Type | Ingredient | (w/w) | (mg/serving) |
| Human milk | 3′-SL or 6′-SL or | 1%-80% | 30 mg-2400 mg |
| oligosaccharide | 2′-FL | | |

TABLE XXXI
(Formula 26)

| | | Wt % | Amount |
|---|---|---|---|
| Type | Ingredient | (w/w) | (mg/serving) |
| Human milk | 3′-SL or 6′-SL | 1%-80% | 30 mg-2400 mg |
| oligosaccharide | or 2′-FL | | |
| Antioxidant/Polyphenol | Pomegranate | 1%-60% | 30 mg-1800 mg |
| | Extract | | |

TABLE XXXII
(Formula 27)

| | | Wt % | Amount |
|---|---|---|---|
| Type | Ingredient | (w/w) | (mg/serving) |
| Human milk | 3′-SL or 6′-SL | 1%-80% | 30 mg-2400 mg |
| oligosaccharide | or 2′-FL | | |
| Glycocalyx Precursor | Sialic Acid | 1%-80% | 30 mg-2400 mg |

TABLE XXXIII
(Formula 28)

| | | Wt % | Amount |
|---|---|---|---|
| Type | Ingredient | (w/w) | (mg/serving) |
| Human milk | 3′-SL or 6′-SL | 1%-60% | 30 mg-1800 mg |
| oligosaccharide | or 2′-FL | | |
| Antioxidant/Polyphenol | Pomegranate | 1%-60% | 30 mg-1800 mg |
| | Extract | | |
| Glycocalyx Precursor | Sialic Acid | 1%-60% | 30 mg-1800 mg |

TABLE XXXIV
(Formula 29)

| | | Wt % | Amount |
|---|---|---|---|
| Type | Ingredient | (w/w) | (mg/serving) |
| Human milk | 3′-SL | 1%-80% | 30 mg-2400 mg |
| oligosaccharide | | | |
| Human milk | 6′-SL | 1%-80% | 30 mg-2400 mg |
| oligosaccharide | | | |
| Human milk | 2′-FL | 0%-80% | 0 mg-2400 mg |
| oligosaccharide | | | |

TABLE XXXV
(Formula 30)

| | | Wt % | Amount |
|---|---|---|---|
| Type | Ingredient | (w/w) | (mg/serving) |
| Human milk | 3′-SL | 1%-80% | 30 mg-2400 mg |
| oligosaccharide | | | |
| Human milk | 6′-SL | 1%-80% | 30 mg-2400 mg |
| oligosaccharide | | | |
| Human milk | 2′-FL | 1%-80% | 30 mg-2400 mg |
| oligosaccharide | | | |
| Antioxidant/ | Pomegranate | 1%-40% | 30 mg-1200 mg |
| Polyphenol | Extract | | |
| Glycocalyx Precursor | Sialic Acid | 1%-40% | 30 mg-1200 mg |

### Example 11: Human Milk Oligosaccharide (hMO) and Chito-Oligosaccharide (COS) Compositions

Compositions are prepared using the components set forth in Table XXXVI-XXXXII.

TABLE XXXVI
(Formula 31)

| | | Wt % | Amount |
|---|---|---|---|
| Type | Ingredient | (w/w) | (mg/serving) |
| Human milk | 3′ -SL or 6′-SL or | 1%-80% | 30 mg-2400 mg |
| oligosaccharide | 2′-FL | | |
| Chito-oligosaccharide | COS | 1%-80% | 30 mg-2400 mg |

TABLE XXXVII
(Formula 32)

| | | Wt % | Amount |
|---|---|---|---|
| Type | Ingredient | (w/w) | (mg/serving) |
| Human milk | 3′-SL or 6′-SL | 1%-40% | 30 mg-1200 mg |
| oligosaccharide | or 2′-FL | | |
| Chito-oligosaccharide | COS | 1%-40% | 30 mg-1200 mg |
| Antioxidant/Polyphenol | Pomegranate | 1%-40% | 30 mg-1200 mg |
| | Extract | | |

TABLE XXXVIII
(Formula 33)

| | | Wt % | Amount |
|---|---|---|---|
| Type | Ingredient | (w/w) | (mg/serving) |
| Human milk | 3′-SL or 6′-SL | 1%-60% | 30 mg-1800 mg |
| oligosaccharide | or 2′-FL | | |
| Chito-oligosaccharide | COS | 1%-60% | 30 mg-1800 mg |
| Glycocalyx Precursor | Sialic Acid | 1%-60% | 30 mg-1800 mg |

TABLE XXXIX
(Formula 34)

| | | Wt % | Amount |
|---|---|---|---|
| Type | Ingredient | (w/w) | (mg/serving) |
| Human milk | 3′-SL or 6′-SL | 1%-60% | 30 mg-1800 mg |
| oligosaccharide | or 2′-FL | | |
| Chito-oligosaccharide | COS | 1%-60% | 30 mg-1800 mg |
| Antioxidant/Polyphenol | Pomegranate | 1%-60% | 30 mg-1800 mg |
| | extract | | |
| Glycocalyx Precursor | Sialic Acid | 1%-60% | 30 mg-1800 mg |

TABLE XXXX
(Formula 35)

| | | Wt % | Amount |
|---|---|---|---|
| Type | Ingredient | (w/w) | (mg/serving) |
| Human milk | 3′-SL | 1%-80% | 30 mg-2400 mg |
| oligosaccharide | | | |
| Human milk | 6′-SL | 1%-80% | 30 mg-2400 mg |
| oligosaccharide | | | |
| Chito-oligosaccharide | COS | 1%-80% | 30 mg-2400 mg |

TABLE XXXXI
(Formula 36)

| | | Wt % | Amount |
|---|---|---|---|
| Type | Ingredient | (w/w) | (mg/serving) |
| Human milk | 3′-SL | 1%-80% | 30 mg-2400 mg |
| oligosaccharide | | | |
| Human milk | 6′-SL | 1%-80% | 30 mg-2400 mg |
| oligosaccharide | | | |
| Chito-oligosaccharide | COS | 1%-80% | 30 mg-2400 mg |
| Antioxidant/Polyphenol | Pomegranate | 0%-50% | 0 mg-1500 mg |
| | extract | | |
| Glycocalyx Precursor | Sialic Acid | 0%-50% | 0 mg-1500 mg |

TABLE XXXXII
(Formula 37)

| | | Wt % | Amount |
|---|---|---|---|
| Type | Ingredient | (w/w) | (mg/serving) |
| Human milk | 3′-SL | 1%-80% | 30 mg-2400 mg |
| oligosaccharide | | | |
| Human milk | 6′-SL | 1%-80% | 30 mg-2400 mg |
| oligosaccharide | | | |
| Human milk | 2′-FL | 1%-80% | 30 mg-2400 mg |
| oligosaccharide | | | |
| Chito-oligosaccharide | COS | 1%-80% | 30 mg-2400 mg |
| Antioxidant/Polyphenol | Pomegranate | 0%-50% | 0 mg-1500 mg |
| | extract | | |
| Glycocalyx Precursor | Sialic Acid | 0%-50% | 0 mg-1500 mg |

It is understood that the above-described various types of compositions, dosage forms and/or modes of applications are only illustrative of preferred embodiments of the present disclosure. Numerous modifications and alternative arrangements may be devised by those skilled in the art without departing from the spirit and scope of the present disclosure and the appended claims are intended to cover such modifications and arrangements. Thus, while the present disclosure has been described above with particularity and detail in connection with what is presently deemed to be the most practical and preferred embodiments of the disclosure, it will be apparent to those of ordinary skill in the art that variations including, but not limited to, variations in size, quantities, materials, shape, form, function and manner of operation, assembly and use may be made without departing from the principles and concepts set forth herein.

What is claimed is:

1. A method of treating intestinal glycocalyx dysfunction in a subject, comprising:
identifying intestinal glycocalyx dysfunction in the subject; and
administering to the subject a chito-oligosaccharide (COS) in an amount and at a frequency sufficient to prevent, stabilize, and reverse damage in an intestinal glycocalyx.

2. The method of claim 1, wherein at least one of:
a. the COS is a chitosan having a polymerization of less than or equal to 50 and an average molecular weight of less than 10 kD;
b. the COS is administered to the subject in an amount of from about 50 mg to about 2500 per dose; or
c. the COS is administered to the subject at a frequency of 1 to 5 times daily.

3. The method of claim 1, wherein identifying comprises at least one of biomarkers, light/electron microscopy of intestinal tissue biopsies, intestinal permeability assay, confocal laser endomicroscopy, or a combination thereof.

4. The method of claim 1, wherein intestinal glycocalyx dysfunction comprises at least one of inflammatory bowel disease, irritable bowel syndrome, celiac disease, small intestinal bacterial overgrowth (SIBO), leaky gut syndrome, colon cancer, or a combination thereof.

5. The method of claim 1, wherein the COS has a degree of polymerization of less than or equal to 50, an average molecular weight of not more than 10 kD, and a degree of deacetylation of 10% to 95%.

6. The method of claim 1, further comprising: administering a rhamnan sulfate wherein at least one of:
a. the rhamnan sulfate has an average molecular weight (MW) of greater than 10 kDa;
b. the rhamnan sulfate is administered in an amount of from about 10 mg to about 1000 mg per dose; or
c. the rhamnan sulfate is administered at a frequency of 1 to 5 times daily.

7. The method of claim 1, further comprising: administering a human milk oligosaccharide (hMO), wherein at least one of:
a. the hMO is a member selected from the group consisting of neutral hMO, neutral N-containing hMO, acid hMO, and a combination thereof;
b. the hMO is administered to the subject in an amount of from about 10 mg to about 5,000 mg per dose; or
c. the hMO is administered to the subject at a frequency of 1 to 5 times daily.

8. The method of claim 1, further comprising: administering an intestinal glycocalyx precursor to the subject wherein at least one of:
a. the intestinal glycocalyx precursor is at least one of: sialic acid, glucosamine, hyaluronic acid, chondroitin sulfate, heparan sulfate, dermatan sulfate, and a combination thereof;
b. the intestinal glycocalyx precursor is administered to the subject in an amount of from about 10 mg to about 5,000 mg per dose; or
c. the intestinal glycocalyx precursor is administered to the subject at a frequency of 1 to 5 times daily.

9. The method of claim 1, further comprising administering a(n) antioxidant/polyphenol to the subject, wherein at least one of:
a. the antioxidant/polyphenol is administered to the subject at a frequency of 1 to 5 times daily; or
b. the antioxidant/polyphenol is administered to the subject in an amount of from about 50 mg to about 5000 mg per dose.

10. The method of claim 1, wherein the amount and the frequency are sufficient to maintain at least one of an intestinal glycocalyx thickness, an intestinal glycocalyx length, an intestinal glycocalyx volume, an intestinal glycocalyx integrity, an intestinal glycocalyx function, or a combination thereof.

11. The method of claim 1, wherein the administering is performed orally.

12. The method of claim 1, wherein the administering is performed via injection.

13. A therapeutic composition for treating intestinal glycocalyx dysfunction, comprising:
a chito-oligosaccharide (COS) in an amount sufficient to maintain an intestinal glycocalyx; and
a pharmaceutically acceptable carrier.

14. The therapeutic composition of claim 13, wherein at least one of:
a. the COS is present in the composition in an amount of from greater than 0 wt % to about 90 wt %;
b. the COS is a chitosan having a polymerization of less than or equal to 50, an average molecular weight of less than 10 kD, and a degree of deacetylation of 10% to 95%; or
c. the COS is present in the composition in an amount of from about 10 wt % to about 100 wt %.

15. The therapeutic composition of claim 13, further comprising a rhamnan sulfate wherein at least one of:
a. the rhamnan sulfate has an average molecular weight (MW) of less than 500 kDa; or
b. the rhamnan sulfate is present in the composition in an amount of from greater than 0 wt % to about 90 wt %.

16. The therapeutic composition of claim 13, further comprising a human milk oligosaccharide (hMO), wherein at least one of:
a. the hMO is a member selected from the group consisting of neutral or fucosylated hMO, neutral N-containing or nonfucosalated hMO, acid or sialylated hMO, and a combination thereof; or
b. the hMO is present in the composition in an amount of from greater than 0 wt % to about 90 wt %.

17. The therapeutic composition of claim 13, further comprising an intestinal glycocalyx precursor, wherein at least one of:
a. the intestinal glycocalyx precursor is at least one of: sialic acid, glucosamine, hyaluronic acid, chondroitin sulfate, heparan sulfate, dermatan sulfate, and a combination thereof; or
b. the glycocalyx precursor is present in the composition in an amount of from about 10 wt % to about 90 wt %.

18. The therapeutic composition of claim 13, further comprising a(n) antioxidant/polyphenol.

19. The therapeutic composition of claim 18, wherein the antioxidant/polyphenol is fermented pomegranate.

20. The therapeutic composition of claim 13, wherein the pharmaceutically acceptable carrier comprises at least one of water, a solubilizing agent, a dispersing agent, a tonicity agent, a pH adjuster, a buffering agent, a preservative, a chelating agent, a bulking agent, a binder, a disintegrant, a filler, a glidant, a lubricant, a sweetener, a thickening agent, or a combination thereof.

21. The therapeutic composition of claim 13, wherein the amount is sufficient to maintain at least one of an intestinal glycocalyx thickness, an intestinal glycocalyx length, an intestinal glycocalyx volume, an intestinal glycocalyx integrity, an intestinal glycocalyx function, or a combination thereof.

22. The therapeutic composition of claim 13, wherein the therapeutic composition is formulated as an oral dosage form.

23. The therapeutic composition of claim 13, wherein the therapeutic composition is formulated as a parenteral dosage form.
